SEQUENCE LISTING (1 sequence)

SEQ ID NO: 1 - protein, 127 aa, Homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

(19) United States
(12) Patent Application Publication
(10) Pub. No.: US 2023/0221337 A1
(43) Pub. Date: Jul. 13, 2023

(54) METHOD FOR TESTING AGGRAVATION RISK OF PERSON INFECTED WITH NOVEL CORONAVIRUS, TEST KIT THEREFOR, COMPANION DIAGNOSTIC DRUG AND AGGRAVATION RISK MARKER THEREOF

(71) Applicants: NATIONAL CENTER FOR GLOBAL HEALTH AND MEDICINE, Tokyo (JP); TIMEWELL MEDICAL Co., Ltd., Tokyo (JP)

(72) Inventors: Daisuke KATAGIRI, Tokyo (JP); Eisei NOIRI, Tokyo (JP); Norio OHMAGARI, Tokyo (JP); Takeshi SUGAYA, Tokyo (JP)

(73) Assignees: NATIONAL CENTER FOR GLOBAL HEALTH AND MEDICINE, Tokyo (JP); TIMEWELL MEDICAL Co., Ltd., Tokyo (JP)

(21) Appl. No.: 17/928,371

(22) PCT Filed: Jun. 18, 2020
(86) PCT No.: PCT/JP2020/023982

§ 371 Date: Nov. 29, 2022

(51) Int. Cl. G01N 33/68

(52) U.S. Cl. CPC G01N 33/6893; G01N 2800/26; G01N 2800/52

(57) ABSTRACT

The purpose of the present invention is to provide: a method for testing the aggravation risk for a person infected with the COVID-19 virus, the method allowing the use of urine as a test sample; a test kit for the method; and a companion diagnostic drug and an aggravation risk marker thereof. The present invention is a method that includes a step for determining the amount of liver fatty acid binding protein in urine sampled from a subject, and that tests the aggravation risk of SARS-CoV-2 infectious disease (COVID-19) on the basis of the quantitative determination result.

### TECHNICAL FIELD

The present invention relates to: a method of assessing an aggravation risk (risk of increase in severity) in a person infected with a novel coronavirus (e.g., SARS-CoV-2); an assessment kit for use in such a method; a companion diagnostic agent; and a marker of the aggravation risk.

### BACKGROUND ART

The number of options for determining the presence or absence of infection with the novel coronavirus (hereinafter also simply referred to as “SARS-CoV-2”) is increasing, such as PCR, antibody testing, and antigen testing. However, a need exists for an assessment method that helps to predict aggravation at an early stage without any complicated operation, since some test-positive patients under watch-and-wait experience a rapid aggravation, which makes watchful treatment difficult.

The liver-type fatty acid binding protein (L-type fatty acid binding protein (hereinafter also simply referred to as “L-FABP”) exists in cytoplasm of the liver and the proximal tubule of the kidneys, etc. The kidneys increase urinary excretion of L-FABP in response to ischemia or oxidative stress caused by renal tubular injury (see, for example, Non-Patent Document 1). This allows kidney diseases to be detected on the basis of detection of the total amount of urinary L-FABP derived from kidney tissues (see, for example, Patent Documents 1 and 2). L-FABP has physiological characteristics that correlate with microhemodynamics. In the conventional art, urinary L-FABP is known as an indicator of renal tubular damage, such as acute kidney injury (AKI) (see, for example, Non-Patent Documents 2 to 5).
Patent Document 1: Japanese Unexamined Patent Application, Publication No. H11-242026
Patent Document 2: Japanese Patent No. 5565607
Non-Patent Document 1: Kamijo, A. et al.: J Lab Clin Med, 143:23-30, 2004
Non-Patent Document 2: Doi K, et al.: Evaluation of new acute kidney injury biomarkers in a mixed intensive care unit. Crit Care Med. 39(11):2464-2469, 2011
Non-Patent Document 3: Doi K, et al.: Lung injury following acute kidney injury: kidney-lung crosstalk. Clin Exp Nephrol. 15(4):464-470, 2011
Non-Patent Document 4: Ishii T, et al.: Neutrophil elastase contributes to acute lung injury induced by bilateral nephrectomy. Am J Pathol. 177(4):1665-1673, 2010
Non-Patent Document 5: Doi K, et al.: Urinary L-type fatty acid-binding protein as a new renal biomarker in critical care. Curr Opin Crit Care. 16(6):545-459, 2010

### DISCLOSURE OF THE INVENTION

### Problems to be Solved by the Invention

Concerning aggravation of COVID-19, a relationship has been reported between systemic microvascular endothelial inflammation and ischemia, thrombosis, necrotic lesions, or other symptoms. Unfortunately, no marker is known that can reflect, in advance, a pathological condition capable of inducing aggravation of COVID-19 and can be detected in specimens collected with no risk of exposure to SARS-CoV-2-containing droplets.

The present invention has been made in view of the conventional technical circumstances shown above. It is an object of the present invention to provide: a method capable of assessing the aggravation risk in a COVID-19-infected person using urine as a specimen; an assessment kit for use in such a method; a companion diagnostic agent; and a marker of the aggravation risk.

### Means for Solving the Problems

As a result of intensive studies for solving the problem shown above, the inventors have found that the potential aggravation risk can increase with increasing urinary L-FABP concentration and that the urinary L-FABP concentration is useful for discriminating the aggravation risk of COVID-19 regardless of renal damage. The present invention has been completed based on the findings. Specifically, the present invention has the following aspects.

(1) A method of assessing the aggravation risk of SARS-CoV-2 infectious disease (COVID-19), the method comprising: quantifying a liver-type fatty acid binding protein in urine collected from a subject; and assessing the aggravation risk of SARS-CoV-2 infectious disease (COVID-19) based on the result of the quantification.
(2) The method according to aspect (1), wherein the quantification is performed at least 2 times at specific intervals of days.
(3) A COVID-19 aggravation risk assessment kit for use in the method according to aspect (1) or (2), the kit comprising a material for quantifying a liver-type fatty acid binding protein.
(4) The COVID-19 aggravation risk assessment kit according to aspect (3), wherein the material for quantifying a liver-type fatty acid binding protein is an anti-L-FABP antibody.
(5) A companion diagnostic agent comprising a material for quantifying a liver-type fatty acid binding protein, the companion diagnostic agent being for use in selecting a therapeutic or prophylactic drug for COVID-19 using the method according to aspect (1) or (2).
(6) A COVID-19 aggravation risk marker comprising a liver-type fatty acid binding protein and being for use as a target to be quantified in the method according to aspect (1) or (2).

### Effects of the Invention

The present invention makes it possible to use urine as a specimen for aggravation risk assessment of COVID-19, which can be collected with no risk of exposure to SARS-CoV-2-containing droplets. The present invention provides a noninvasive method that helps to assess the aggravation risk of COVID-19 at an early stage. The present invention also makes it possible to perform early-stage triage of aggravation risk (risk classification), for example, in the case of watchful waiting of SARS-CoV-2-positive patients. The present invention also makes it possible to provide an L-FABP point-of-care (POC) kit having high accuracy for identifying only patients at a high aggravation risk, which will contribute to medical resource optimization to address acute deterioration of positive waiting list patients and to prepare for transfer of such patients to intensive care unit (ICU) at appropriate time. The present invention also makes it possible to assess the risk of recurrence after returning home with respect to patients waiting for discharge (including patients who return home after an approximately two-week wait at a neighboring facility).

### BRIEF DESCRIPTION OF THE DRAWINGS

FIGS. 1(a) and 1(b) are graphs showing the results of ROC analysis of serum creatinine concentrations (mg/dL) on admission for discrimination of the degree of severity at one week after admission; FIGS. 1(c) and 1(d) are graphs showing the results of ROC analysis of urinary L-FABP concentrations (ng/mL) on admission for discrimination of the degree of severity at one week after admission; FIG. 1(e) is a graph showing serum creatinine concentration (mg/dL) on admission and progression of the degree of severity; FIG. 1(f) is a graph showing urinary L-FABP concentration (ng/mL) on admission and progression of the degree of severity;

FIG. 2 is a two-dimensional plot graph showing whether the aggravation risk of SARS-CoV-2 correlates with urinary L-FABP concentration or serum creatinine concentration;

FIGS. 3(a) and 3(b) are graphs showing the results of ROC analysis of serum creatinine concentrations (mg/dL) on admission for discrimination of the degree of severity at one week after admission; FIGS. 3(c) and 3(d) are graphs showing the results of ROC analysis of urinary L-FABP concentrations (μg/gCre) on admission corrected with urinary creatinine concentration for discrimination of the degree of severity at one week after admission; FIG. 3(e) is a graph showing serum creatinine concentration (mg/dL) on admission and progression of the degree of severity; FIG. 3(f) is a graph showing progression of the degree of severity and urinary L-FABP concentration (μg/gCre) on admission corrected with urinary creatinine concentration;

FIG. 4 is a two-dimensional plot graph showing whether the aggravation risk of SARS-CoV-2 correlates with urinary L-FABP concentration (corrected with urinary creatinine concentration) or serum creatinine concentration;

FIGS. 5(a) to 5(c) are graphs in which with respect to 58 SARS-CoV-2-positive patients, the serum creatinine concentration (mg/dL) on admission, the urinary N-acetyl-β-D-glucosaminidase (NAG) concentration (U/L) on admission, and the urinary L-FABP concentration (μg/gCre) on admission corrected with urinary creatinine concentration are each plotted against the number of days from onset to admission (to the measurement of concentration);

FIGS. 6(a) to 6(c) are graphs showing correlation between the marker levels measured on admission of 41 mild patients and the pathological progression in one week;

FIGS. 7(a) and 7(b) are a graph and a table showing the results of ROC analysis of the creatinine-corrected urinary L-FABP concentrations on admission (and the serum creatinine concentrations for comparison) of “mild-moderate” and “mild-severe” patients versus “mild-mild” patients; and

FIGS. 8(a) and 8(b) are a graph and a table showing the results of ROC analysis of the urinary L-FABP concentrations on admission (and the serum creatinine concentrations for comparison) of “mild-moderate” and “mild-severe” patients versus “mild-mild” patients.

### PREFERRED MODE FOR CARRYING OUT THE INVENTION

Hereinafter, embodiments of the present invention will be described in detail, which are not intended to limit the present invention and may be altered or modified as needed for implementation without departing from the gist of the present invention.

### L-FABP

The L-FABP amino acid sequence and the gene sequence for L-FABP have already been reported (Veerkamp and Maatman, Prog. Lipid Res., 34:17-52, 1995). SEQ ID NO: 1 represents the amino acid sequence of the wild-type human liver-type fatty acid binding protein (L-FABP). The term “liver-type fatty acid binding protein (L-FABP)” is intended to include all mutants of the wild-type human L-FABP of SEQ ID NO: 1 in the Sequence Listing, which have a deletion, substitution, and/or addition of one or more amino acids with respect to the amino acid sequence of SEQ ID NO: 1, as long as the three-dimensional structure of such mutants is highly conserved with respect to that of the wild-type human L-FABP of SEQ ID NO: 1. Different amino acid side chains for serving as protein components have different hydrophobic properties, electric charges, sizes, or other properties. It is known empirically or from physicochemical measurements that some amino acids can be substituted for other amino acids without substantially affecting the three-dimensional structure (also referred to as “conformation”) of a protein as a whole, in other words, in a highly conservative way. Examples of such conservative substitutions of amino acid residues include those between glycine (Gly) and proline (Pro), Gly and alanine (Ala) or valine (Val), leucine (Leu) and isoleucine (Ile), glutamic acid (Glu) and glutamine (Gln), aspartic acid (Asp) and asparagine (Asn), cysteine (Cys) and threonine (Thr), Thr and serine (Ser) or Ala, and lysine (Lys) and arginine (Arg).

The L-FABP may be obtained by any method. The L-FABP may be a chemically synthesized protein or a recombinant protein produced by genetic recombination techniques.

Method of Assessing the Aggravation Risk of COVID-19 (hereinafter also simply referred to as “the assessment method according to the first embodiment”) A first embodiment of the present invention is directed to a method comprising: quantifying L-FABP in urine collected from a subject (e.g., a patient); and assessing a potential aggravation risk of COVID-19 based on the result of the quantification. As shown in the examples below, the urinary L-FABP concentration can increase with increasing potential aggravation risk, regardless of the renal damage-induced increase in urinary marker level or to such an extent as to significantly exceed the renal damage-induced increase in urinary marker level. The subject may have been confirmed infected with SARS-CoV-2 or suffering from COVID-19. The subject preferably has a COVID-19 condition before suffering from aggravation (including a mild or no symptom case) or a moderate COVID-19 condition and more preferably has a mild COVID-19 condition. The subject from which urine is collected for the quantification is preferably within 20 days from the disease onset, more preferably within 14 days from the disease onset, even more preferably within 12 days from the disease onset, and most preferably within 10 days from the disease onset. The lower limit of the number of days from the disease onset should not be limited. The lower limit of the number of days from the disease onset may be zero, one or more, or two or more. Moreover, the subject may or may not have experienced the disease onset. As used herein, the term “disease onset” refers to the development of a condition, examples of which include mild, moderate, and severe conditions, which may be classified on the 8-category scale shown below (categories (1) to (8)).

The degree of severity of COVID-19 may be, for example, mild, moderate, or severe. The aggravation of COVID-19 may be aggravation from mild to moderate or severe or aggravation from moderate to severe. In the description and claims, the degree of severity, such as mild, moderate, and severe, is classified on the 8-category scale (categories (1) to (8) shown below) shown in Cao B. Wang Y, Wen D, et al. A Trial of Lopinavir-Ritonavir in Adults Hospitalized with Severe Covid-19. N Engl J Med. 2020.

(1) Not hospitalized due to resumption of usual activities;
(2) Not hospitalized, but unable to resume normal activities;
(3) Hospitalization, not requiring supplemental oxygen;
(4) Hospitalization, requiring supplemental oxygen;
(5) Hospitalization, requiring high-flow nasal cannula oxygen therapy (HFNC), noninvasive mechanical ventilation, or both;
(6) Hospitalization, requiring invasive mechanical ventilation;
(7) Hospitalization, requiring mechanical ventilation and extracorporeal membrane ventilation (ECMO); and

### (8) Death

Specifically, in the description and claims, the term “mild” is intended to include categories (1) to (3), the term “moderate” is intended to include categories (4) and (5), and the term “severe” is intended to include categories (6) to (8).

The subject with a urinary L-FABP concentration (ng/mL) higher than or equal to a specific value (cutoff value or pathological discrimination value) may be identified as having an aggravation risk or having a high potential aggravation risk. The urinary L-FABP concentration may or may not be a urinary creatinine-corrected urinary L-FABP concentration (μg/gCre). According to the present invention, the aggravation risk of COVID-19 can be simply assessed only based on the urinary L-FABP concentration not corrected with urinary creatinine concentration using a simple tool, such as a POC kit. The concentration of urinary components may vary greatly depending on urine concentration. In the present invention, the urinary L-FABP concentration may be corrected with urinary creatinine concentration, and the urinary creatinine-corrected L-FABP concentration (μg/gCre) may be used so that the aggravation risk of COVID-19 can be assessed with higher accuracy with no influence of urine concentration. In view of assessment accuracy, the cutoff value (pathological discrimination value) at which the subject with a mild or moderate condition is determined to have a potential aggravation risk or a potential high aggravation risk is preferably in the range of 35 ng/mL to 40 ng/mL, more preferably in the range of 36 ng/mL to 39 ng/mL, even more preferably in the range of 37 ng/mL to 38 ng/mL. In a case where the urinary L-FABP concentration is corrected (μg/gCre) with urinary creatinine concentration, the cutoff value is preferably in the range of 18 μg/gCre to 25 μg/gCre, more preferably in the range of 20 μg/gCre to 24 μg/gCre, even more preferably in the range of 21 μg/gCre to 23 μg/gCre. If the cutoff value is lower than the lower limit shown above, the number of false-positive cases may increase. If the cutoff value is higher than the upper limit shown above, there may be a risk of missing patients having an aggravation risk of developing a severe condition.

The subject with a mild condition and with a urinary L-FABP concentration (ng/mL) lower than or equal to a specific level (cutoff value) may be identified as having no potential aggravation risk or a low potential aggravation risk. The urinary L-FABP concentration may or may not be a urinary creatinine-corrected urinary L-FABP concentration (μg/gCre). The cutoff value for identifying the subject with a mild condition as likely remaining unchanged or as having no potential aggravation risk or a low potential aggravation risk is typically in the range of 35 ng/mL to 30 ng/mL, preferably in the range of 34 ng/mL to 31 ng/mL, more preferably in the range of 33 ng/mL to 32 ng/mL. In a case where the urinary L-FABP concentration is corrected (μg/gCre) with urinary creatinine concentration, the cutoff value is typically in the range of 12 μg/gCre to 7 μg/gCre, preferably in the range of 11 μg/gCre to 8 μg/gCre, more preferably in the range of 10 μg/gCre to 9 μg/gCre. If the cutoff value is higher than the upper limit shown above, there may be a high risk of missing patients having an aggravation risk. If the cutoff value is lower than the lower limit shown above, there may be a high risk of missing patients having no aggravation risk.

The cutoff value for identifying the subject with a mild condition as having a potential aggravation risk or a potential high aggravation risk from mild to moderate or severe is preferably in the range of 30 ng/mL to 35 ng/mL, more preferably in the range of 31 ng/mL to 34 ng/mL, even more preferably in the range of 32 ng/mL to 33 ng/mL. In a case where the urinary L-FABP concentration is corrected (μg/gCre) with urinary creatinine concentration, the cutoff value is preferably in the range of 9 μg/gCre to 14 μg/gCre, more preferably in the range of 10 μg/gCre to 13 μg/gCre, even more preferably in the range of 11 μg/gCre to 12 μg/gCre. If the cutoff value is lower than the lower limit shown above, the number of false-positive cases may increase. If the cutoff value is higher than the upper limit shown above, there may be a risk of missing patients having an aggravation risk of from mild to moderate or severe.

The cutoff value for identifying the subject with a mild condition as having no potential aggravation risk or a low aggravation risk of from mild to moderate or severe is typically in the range of 34 ng/mL to 30 ng/mL, preferably in the range of 33 ng/mL to 31 ng/mL, more preferably in the range of 32.5 ng/mL to 31.5 ng/mL. In a case where the urinary L-FABP concentration is corrected (μg/gCre) with urinary creatinine concentration, the cutoff value is typically in the range of 14 μg/gCre to 9 μg/gCre, preferably in the range of 12 μg/gCre to 10 μg/gCre, more preferably in the range of 11.5 μg/gCre to 10.5 μg/gCre. If the cutoff value is higher than the upper limit shown above, the number of false-negative cases may increase. If the cutoff value is lower than the lower limit shown above, there may be a risk of missing patients having no aggravation risk.

The aggravation risk of COVID-19 any days ahead may be predicted. For example, the aggravation risk of COVID-19 one or more days ahead of the quantification may be predicted. For prediction more days ahead, the aggravation risk of COVID-19 preferably two or more days ahead (more preferably 3 or more days ahead, even more preferably 4 or more days ahead, furthermore preferably 5 or more days ahead, still more preferably 6 or more days ahead, most preferably 7 or more days ahead) of the quantification is predicted. The upper limit of the number of days ahead for the prediction is typically, but not limited to, 30 or less, 20 or less, or 15 or less.

As mentioned above, the subject preferably has a mild condition for the purpose of monitoring pathological progression, pathological course, or aggravation progression. For the purpose of monitoring pathological progression, pathological course, or aggravation progression, the quantification is preferably performed multiple times (preferably at least 2 times, more preferably 3 or more times, even more preferably 4 or more times) at specific intervals of days. The upper limit of the number of times of the quantification is typically, but not limited to, 15 or less or 10 or less. The quantification may be performed multiple times at intervals of any days. The quantification may be performed (once) every 3 or more days, preferably every 4 or more days, even more preferably every 5 or more days, most preferably every 7 or more days. The intervals of days between the multiple quantification operations may have any upper limit. For example, the quantification may be performed once every 2 or 3 weeks.

The method of assessing the aggravation risk of COVID-19 according to the first embodiment may or may not include collecting urine from the subject. The assessment method according to the first embodiment may or may not include detecting L-FABP in urine. In the assessment method according to the first embodiment, the method for detecting, quantifying, or measuring L-FABP may be enzyme immunoassay (EIA, ELISA), fluorescence enzyme immunoassay (FLEIA), chemiluminescence enzyme immunoassay (CLEIA), chemiluminescence immunoassay (CLIA), electrochemiluminescence immunoassay (ECLIA), fluorescent antibody assay (FA), radioimmunoassay (RIA), Western blotting (WB), immunoblotting, or any other assay. L-FABP is preferably detected, quantified, or measured by a method using an anti-L-FABP antibody.

The anti-L-FABP antibody may be any type capable of recognizing L-FABP. The anti-L-FABP antibody may be a known antibody or one developed in the future. For example, the anti-L-antibody may be one capable of recognizing a moiety that has been exposed to outside by the denaturation shown below.

L-FABP in blood may be denatured with a surfactant and then subjected to the quantification using the anti-L-FABP antibody. In this case, the denatured L-FABP has a modified three-dimensional structure with hydrogen bonds and disulfide bonds cleaved and with the primary structure remaining intact. The denatured L-FABP can be detected or quantified in a highly sensitive and specific manner without influence of an oxidized state of L-FABP even in a case where the antibody binds to an inner region of the L-FABP molecule. The surfactant is preferably sodium dodecyl sulfate (SDS). The denaturation process may include treatment with the surfactant at a suitable concentration (e.g., 0.2 to 10% by mass/volume (w/v %), preferably 0.4% by mass/volume (w/v %) or more, 0.5% by mass/volume (w/v %) or more, or 0.7% by mass/volume (w/v %) or more) at room temperature (e.g., 25° C.) or under heating conditions (e.g., 37° C.) for a suitable period of time (e.g., 5 to 60 minutes). Typically, the denaturation process is performed at 25° C. for 10 minutes using 1 w/v, SDS.

More specifically, the measurement method is preferably sandwich ELISA (enzyme-linked immunosorbent assay) using a combination of two antibodies having different moieties for recognizing the antigen (L-FABP). One of the two antibodies with different recognition moieties is preferably used as a solid-phase antibody bound to the surface of the wells of a microplate, and the other is preferably used as a labeled antibody for detection or quantification. The label of the labeled antibody is typically, but not limited to, an enzyme label, such as a peroxidase label, a fluorescent label, an ultraviolet label, or a radioactive label.

The antibodies with different moieties for recognizing the antigen (L-FABP) may include at least one antibody selected from the group consisting of anti-L-FABP antibody clone 1, clone 2, clone L, and clone F (e.g., those disclosed in Japanese Patent Nos. 6174778, 6218983, and 6059388). A combination including anti-L-FABP antibody clone L or anti-L-FABP antibody clone 2 is preferred, and a combination including anti-L-FABP antibody clone L is more preferred. Even more preferably, anti-L-FABP antibody clone L is used as a solid-phase antibody and any anti-L-FABP antibody is used as a labeled antibody. Most preferably, anti-L-FABP antibody clone L is used as a solid-phase antibody and anti-L-FABP antibody clone 2 is used as a labeled antibody. L-FABP measurement kits based on sandwich ELISA are commercially available, such as Renapro L-FABP Test TMB (manufactured by CMIC Holdings Co., Ltd.) and Renapro L-FABP Test HS (high sensitivity) (manufactured by CMIC Holdings Co., Ltd.).

In the assessment method according to the first embodiment, the quantification step may or may not include: preparing a calibration curve based on the relationship between the measured label intensity (e.g., absorbance, enzyme label intensity, fluorescence intensity, UV intensity, radiation intensity) and the amount (e.g., concentration) of L-FABP; and performing the quantification using the calibration curve (e.g., by comparison).

In view of the accuracy of the assessment method according to the first embodiment, the assessment is preferably carried out with an area under the curve (AUC) of 0.70 or more (70% or more), more preferably 0.80 or more (80% or more), even more preferably 0.85 or more (85% or more) as a result of receiver operating characteristics (ROC) analysis.

The assessment method according to the first embodiment may or may not include a method of diagnosing the aggravation risk of COVID-19. Moreover, the present invention may or may not be directed to a therapeutic or prophylactic method for COVID-19, comprising: performing the COVID-19 aggravation risk assessment method according to the first embodiment; and performing at least one step selected from the group consisting of: performing an action for the subject depending on the aggravation risk determined by the method (preferably the aggravation risk specific days ahead); and administering, to the subject, a therapeutic or prophylactic drug for COVID-19 depending on the aggravation risk determined by the method (preferably the aggravation risk specific days ahead).

When determined to have a low aggravation risk by the method (e.g., when the subject has a mild condition and is determined to still have a mild condition specific days or more ahead by the method), the subject should undergo an action, such as watch-and-wait (e.g., watchful waiting, monitoring), isolation (e.g., home healthcare, home care, accommodation healthcare), administration of commercially available cold medicines (e.g., combination cold remedies, anti-inflammatory analgesics), or hydration.

When determined to have a high aggravation risk by the method (e.g., when determined to have an aggravation risk from mild to moderate or severe or from moderate to severe within specific days by the method), the subject should undergo an action, such as hospitalization, ICU transfer, antibiotic administration against intercurrent microbial infection, oxygen supplementation, high-flow nasal cannula oxygen therapy, noninvasive or invasive mechanical ventilation, extracorporeal membrane oxygenation (ECMO), acute blood purification, or hemoadsorption therapy.

The therapeutic or prophylactic drug for COVID-19 may be at least one drug selected from the group consisting of antiviral drugs (e.g. remdesivir, favipiravir, ciclesonide, ivermectin), cytokine storm- or acute respiratory distress syndrome (ARDS)-ameliorating drugs (e.g., tocilizumab, sarilumab), and vaccines and antibodies (e.g., monoclonal or polyclonal antibodies, preferably antibodies that bind to SARS-CoV-2, more preferably antibodies that selectively bind to SARS-CoV-2, even more preferably antibodies that specifically bind to SARS-CoV-2).

### Assessment Kit, Companion Diagnostic Agent, and COVID-19 Aggravation Risk Marker

A second embodiment of the present invention is directed to a COVID-19 aggravation risk assessment kit comprising a material for quantifying L-FABP. The COVID-19 aggravation risk assessment kit is preferably for use in the assessment method according to the first embodiment and more preferably a POC kit. A third embodiment of the present invention is directed to a companion diagnostic agent comprising a material for quantifying a liver-type fatty acid binding protein. The companion diagnostic agent is preferably for use in selecting an action for addressing the aggravation risk determined by the assessment method according to the first embodiment (preferably the aggravation risk specific days ahead) and/or for use in selecting a therapeutic or prophylactic drug for COVID-19 depending on the aggravation risk. A fourth aspect of the present invention is directed to a COVID-19 aggravation risk marker comprising a liver-type fatty acid binding protein and being for use as a target to be quantified in the method according to the first embodiment. In the description and claims, the term “companion diagnostic agent” refers to a diagnostic agent for use in an assessment that is performed before the start of actual action, medication, or other care in order to predict the effect or risk of the action to be performed on individual COVID-19 patients depending on the determined aggravation risk or in order to predict the effect, side effect risk, or adequate dose of a pharmaceutical (e.g., a therapeutic or prophylactic drug) to be administered to individual COVID-19 patients depending on the determined aggravation risk.

The action to be performed on individual COVID-19 patients depending on the determined aggravation risk may be as mentioned above. The therapeutic or prophylactic drug for COVID-19 may be as mentioned above. The companion diagnostic agent according to the third embodiment is preferably at least one selected from the group consisting of a companion diagnostic agent for predicting the aggravation risk of COVID-19, a companion diagnostic agent for predicting the risk of COVID-19 onset, and a companion diagnostic agent for monitoring the progression of COVID-19 aggravation. The material for quantifying L-FABP for use in the assessment kit according to the second embodiment or for forming the companion diagnostic agent according to the third embodiment may be a material for quantifying L-FABP or oxidized L-FABP on the basis of enzyme immunoassay (EIA, ELISA), fluorescence enzyme immunoassay (FLEIA), chemiluminescence enzyme immunoassay (CLEIA), chemiluminescence immunoassay (CLIA), electrochemiluminescence immunoassay (ECLIA), latex-enhanced immunoturbidimetric assay (LTIA), immunochromatography, fluorescent antibody assay (FA), radioimmunoassay (RIA), Western blotting (WB), immunoblotting, or other assays. Specifically, the material for quantifying L-FABP is preferably an anti-L-FABP antibody.

The anti-L-FABP antibody may be any type capable of recognizing L-FABP. The anti-L-FABP antibody may be a known antibody or one developed in the future. For example, the anti-L-antibody may be one capable of recognizing a moiety that has been exposed to outside by the denaturation shown above, the oxidation of methionine, or other methods.

More specifically, the quantification method is preferably an assay based on sandwich ELISA using a combination of two antibodies having different moieties for recognizing the antigen (L-FABP). The two antibodies having different recognition moieties are as mentioned above in the section “Method of Assessing the Aggravation Risk of COVID-19”.

A reagent or reagents may be used as a means for the quantification. The reagents preferably include the anti-L-FABP antibody and more preferably include a labeled anti-L-FABP antibody. If necessary, the reagents may include an adsorption inhibitor (e.g., bovine serum albumin (BSA), casein, skim milk, polyethylene glycol), a pretreatment liquid (any surfactant, any buffer solution), a reaction buffer (any buffer solution), or a chromogenic substrate (e.g., 3,3′,5,5′-tetramethylbenzidine, hydrogen peroxide water). The content of the adsorption inhibitor in the quantification means is preferably 0.05 to 10% by mass although it should not be limited as long as the advantageous effects of the present invention are not compromised.

The quantification means is preferably a kit for performing sandwich ELISA using a combination of two antibodies having different moieties for recognizing the antigen, in which more preferably, anti-L-FABP antibody clone L is used as a solid-phase antibody and anti-L-FABP antibody clone 2 is used as a labeled antibody.

When for use in the quantification using the anti-L-FABP antibody, the assessment kit according to the second embodiment or the companion diagnostic agent according to the third embodiment preferably includes a means for denaturing L-FABP with a surfactant in advance of the quantification. The assessment kit according to the second embodiment may or may not further comprise: a means for denaturing L-FABP in the urine using a surfactant; and a means for quantifying the denatured L-FABP. The surfactant is as mentioned above. The means for denaturation may include a means for treating L-FABP with the surfactant at any suitable concentration (e.g., 0.2% by mass/volume to 10% by mass/volume) at room temperature (e.g., 25° C.) or under heating conditions (e.g., 37° C.), which may be, for example, denaturation liquids including the surfactant, any buffer solution, and other components.

When based on sandwich ELISA, the assessment kit according to the second embodiment or the companion diagnostic agent according to the third embodiment may be specifically a kit comprising:

(1) an anti-L-FABP antibody-immobilized microplate having wells to which an anti-human L-FABP mouse monoclonal antibody (e.g., derived from a clone L-producing cell line) is bound;
(2) a denaturation liquid (e.g., any surfactant);
(3) a reaction buffer;
(4) an enzyme-labeled antibody (e.g., a peroxidase-labeled anti-human L-FABP mouse monoclonal antibody (e.g., derived from a clone 2-producing cell line));
(5) an enzyme substrate solution;
(6) a detergent (e.g., any buffer solution, surfactant);
(7) a reaction stopping solution (e.g., 1N sulfuric acid);
(8) a standard buffer (any buffer solution); and
(9) an authentic liver-type fatty acid binding protein, in which
(10) the concentration of the authentic liver-type fatty acid binding protein is typically, but not limited to, 10 to 10,000 ng/mL, preferably 50 to 5,000 ng/mL, more preferably 100 to 1,000 ng/mL, even more preferably 200 to 800 ng/mL, and most preferably 300 to 600 ng/mL.

The assessment kit according to the second embodiment or the companion diagnostic agent according to the third embodiment preferably includes a protein preservation buffer containing BSA for preventing protein adsorption. For example, the protein preservation buffer may be as follows.

### Protein Preservation Buffer

A 10 mM phosphate buffer (pH 7.2) containing 150 mM NaCl, 1.0% BSA, and 0.1% NaN₃

### EXAMPLES

Hereinafter, the present invention will be more specifically described with reference to examples, which are not intended to limit the present invention and may be altered or modified in various ways without departing from the technical idea of the present invention.

### Example 1

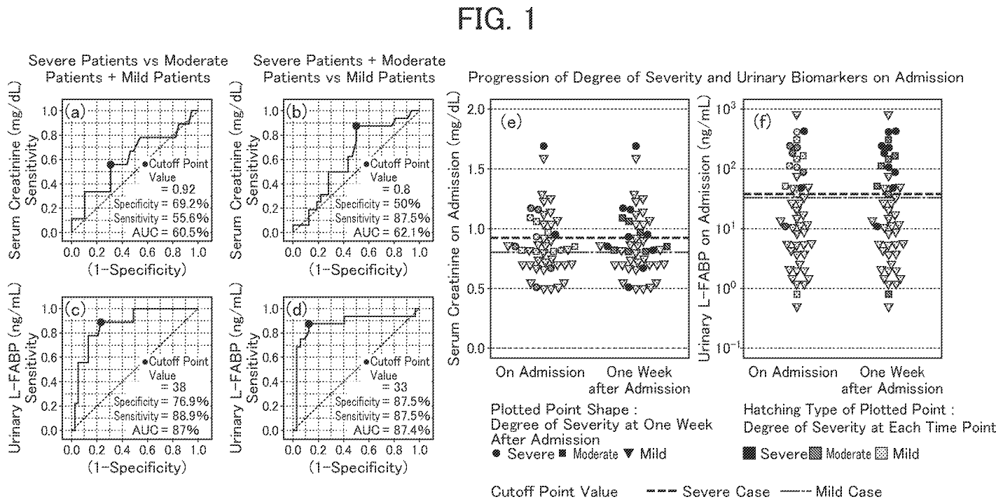

Of 58 SARS-CoV-2-positive patients, 21 mild patients, 25 moderate patients (on oxygen), and 12 severe patients (artificially ventilated) were subjected on admission to measurement of urinary L-FABP concentration (ng/mL) and serum creatinine concentration (mg/dL). The measurements were subjected to ROC analysis for discrimination of a degree of severity at one week after admission. Serum creatinine concentration was measured by the ordinary method. In general, serum creatinine concentration is considered an indicator of renal tissue injury, and serum creatinine concentration increases as glomerular filtration function decreases. Urinary L-FABP concentration (ng/mL) was measured as follows. The urine sample from each patient was subjected to denaturation with 1 w/v % SDS at 25° C. for 10 minutes and then subjected to ELISA measurement using the antibody: Renapro L-FABP Test HS (high sensitivity) (manufactured by CMIC Holdings Co., Ltd.), in which the coloring intensity (OD 450 nm) of the labeled antibody was measured. The measurement using the assay kit was performed according to the attached instructions. The concentration (ng/mL) of L-FABP in the urine was measured. The results are shown in FIGS. 1(a) to 1(d).

FIG. 1(a) is a graph showing the results of ROC analysis of the serum creatinine concentrations (mg/dL) on admission of the patients who were severe one week after admission versus the patients who were moderate or mild one week after admission. The results of ROC analysis shown in FIG. 1(a) indicate that a cutoff point value (pathological discrimination value) of 0.92 (mg/dL) was obtained for the serum creatinine concentrations on admission of the patients who were severe one week after admission, with a specificity of 69.2%, a sensitivity of 55.6%, and an AUC of 60.5%.

FIG. 1(b) is a graph showing the results of ROC analysis of the serum creatinine concentrations (mg/dL) on admission of the patients who were mild one week after admission versus the patients who were severe or moderate one week after admission. The results of ROC analysis shown in FIG. 1(b) indicate that a cutoff point value of 0.8 (mg/dL) was obtained for the serum creatinine concentrations on admission of the patients who were mild one week after admission, with a specificity of 50%, a sensitivity of 87.5%, and an AUC of 62.1%.

FIG. 1(c) is a graph showing the results of ROC analysis of the urinary L-FABP concentrations on admission (ng/mL) of the patients who were severe one week after admission versus the patients who were moderate or mild one week after admission. The results of ROC analysis shown in FIG. 1(c) indicate that a cutoff point value of 38 (ng/mL) was obtained for the urinary L-FABP concentrations (ng/mL) on admission of the patients who were severe one week after admission, with a specificity of 76.9%, a sensitivity of 88.9%, and an AUC of 87%.

FIG. 1(d) is a graph showing the results of ROC analysis of the urinary L-FABP concentrations (ng/mL) on admission of the patients who were mild one week after admission versus the patients who were severe or moderate one week after admission. The results of ROC analysis shown in FIG. 1(d) indicate that a cutoff point value of 33 (ng/mL) was obtained for the urinary L-FABP concentrations (ng/mL) on admission of the patients who were mild one week after admission, with a specificity of 87.5%, a sensitivity of 87.5%, and an AUC of 87.4%.

These results indicate that the AUC, accuracy, sensitivity, and specificity are higher for urinary L-FABP concentration on admission than for serum creatinine concentration on admission both for discrimination of patients who experienced aggravation and for discrimination of patients who did not experience aggravation (remained mild or so) among the SARS-CoV-2-positive patients. It has also been suggested that as the potential aggravation risk increases, the urinary L-FABP concentration should increase regardless of the renal damage-induced increase in urinary marker level or to such an extent as to significantly exceed the renal damage-induced increase in urinary marker level and that the urinary L-FABP concentration will help to assess the potential aggravation risk with no influence of the renal damage-induced increase in urinary marker level. This is unexpected in view of the known relationship between aggravation in viral infection and renal damage. The accuracy in identifying only patients who experienced aggravation was particularly useful. Except for two cases in which AKI developed in 10 days after admission (namely, after the measurement of L-FABP), the persistently high L-FABP concentration was suggested to provide a new useful indicator in COVID-19 aggravation, which is unrelated to AKI.

FIG. 1(e) is a graph showing the serum creatinine concentrations (mg/dL) on admission of the 58 SARS-CoV-2-positive patients, in which two points at the same serum creatinine concentration plotted on the left (on admission) and the right (after one week) are of the same positive patient. In FIG. 1(e), the points of the same positive patient plotted on the left (on admission) and the right (after one week) are both shaped (0: severe, 0: moderate, V: mild) to indicate the degree of severity at one week after admission. In FIG. 1(e), different types of hatching (patterns) are used to distinguish the degree of severity on admission (left) and the severity at one week after admission (right). The change between the degree of severity on admission and that after one week can be understood by comparing the hatching types (patterns) of the points of the same positive patient plotted on the left (on admission) and the right (after one week). In FIG. 1(e), the cutoff point value for severe case is 0.92 (mg/dL) from FIG. 1(a) and that for mild case is 0.8 (mg/dL) from FIG. 1(b). The results shown in FIG. 1(e) suggest no correlation between the serum creatinine concentration on admission and aggravation of SARS-CoV-2.

FIG. 1(f) is a graph showing the urinary L-FABP concentrations (ng/mL) on admission of the 58 SARS-CoV-2-positive patients, in which two points at the same urinary L-FABP concentration plotted on the left (on admission) and the right (after one week) are of the same positive patient. In FIG. 1(f), the points of the same positive patient plotted on the left (on admission) and the right (after one week) are both shaped (0: severe, 0: moderate, V: mild) to indicate the degree of severity at one week after admission. In FIG. 1(f), different types of hatching (patterns) are used to distinguish the degree of severity on admission (left) and the degree of severity at one week after admission (right). The change between the degree of severity on admission and that after one week can be understood by comparing the hatching types (patterns) of the points of the same positive patient plotted on the left (on admission) and the right (after one week). In FIG. 1(f), the cutoff point value for severe case is 38 (ng/mL) from FIG. 1(c) and that for mild case is 33 (ng/mL) from FIG. 1(d). The results plotted in FIG. 1(f) indicate that patients with a urinary L-FABP concentration on admission higher than or equal to a cutoff point value of 38 ng/mL have a significantly strong tendency to experience aggravation from mild to moderate or severe or from moderate to severe in one week. On the other hand, the results plotted in FIG. 1(f) suggest that patients with a urinary L-FABP concentration on admission lower than or equal to a cutoff point value of 33 ng/mL are mild on admission and still mild after one week and have no aggravation risk or a low aggravation risk except for few cases.

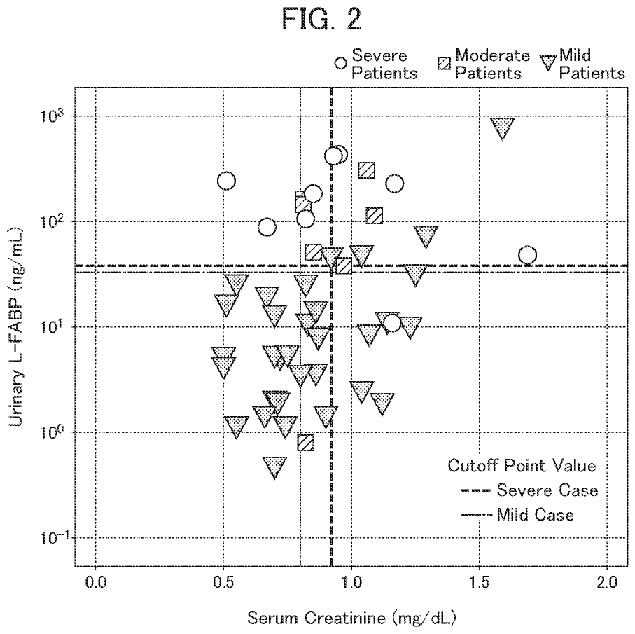

Next, a two-dimensional plot was made in which the urinary L-FABP concentrations on admission of the 58 SARS-CoV-2-positive patients were on the vertical axis with the point shape and the hatching type indicating the degree of severity at one week after admission (corresponding to the left plot in FIG. 1(f)), and the serum creatinine concentrations on admission of the 58 SARS-CoV-2-positive patients were on the horizontal axis with the point shape and the hatching type indicating the degree of severity at one week after admission (corresponding to the right plot in FIG. 1(e)). This further clarified whether the aggravation risk of SARS-CoV-2 correlated with the urinary L-FABP concentration or the serum creatinine concentration. The results are shown in FIG. 2.

The results plotted in FIG. 2 indicate that patients with a urinary L-FABP concentration on admission higher than or equal to a cutoff point value of 38 ng/mL have a significantly strong tendency to experience aggravation in one week. On the other hand, the results plotted in FIG. 2 indicate no correlation between the serum creatinine concentration on admission and aggravation whether or not it exceeds the cutoff point value 0.92 mg/dL.

### Example 2

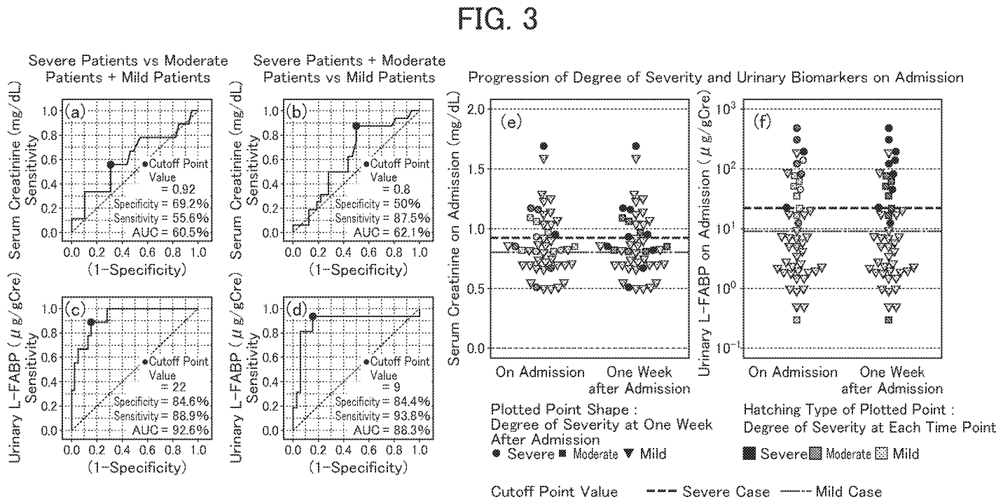

In order to eliminate the influence of urine concentration, the urinary creatinine concentration was used to correct the results of ROC analysis of the urinary L-FABP concentrations of the 58 SARS-CoV-2-positive patients, which were obtained in Example 1, and to correct the urinary L-FABP concentrations on admission. The urinary creatinine concentration was measured by the ordinary method. The results are shown in FIGS. 3(c), 3(d), and 3(f). For comparative reference, FIGS. 3(a) and 3(b) are provided which are the same as FIGS. 1(a) and 1(b) showing the results of ROC analysis of the serum creatinine concentrations (mg/dL), and FIG. 3(e) is provided which is the same as FIG. 1(e) showing the serum creatinine concentrations (mg/dL) on admission and the degree of severity progression. Namely, FIGS. 1(a), 1(b), and 1(e) are the same as FIGS. 3(a), 3(b), and 3(e), respectively.

FIG. 3(c) is a graph showing the results of ROC analysis of the urinary creatinine-corrected urinary L-FABP concentrations (μg/gCre) on admission for discrimination between patients who were severe one week after admission and patients who were moderate or mild one week after admission. The results of ROC analysis shown in FIG. 3(c) indicate that a cutoff point value of 22 (μg/gCre) was obtained for the urinary creatinine-corrected urinary L-FABP concentrations (μg/gCre) on admission of patients who were severe one week after admission, with a specificity of 84.6%, a sensitivity of 88.9%, and an AUC of 92.63.

FIG. 3(d) is a graph showing the results of ROC analysis of the urinary creatinine-corrected urinary L-FABP concentrations (μg/gCre) on admission of patients who were mild one week after admission versus patients who were severe or moderate one week after admission. The results of ROC analysis shown in FIG. 3(d) indicate that a cutoff point value of 9 (μg/gCre) was obtained for the urinary creatinine-corrected L-FABP concentrations (μg/gCre) on admission of patients who were mild one week after admission, with a specificity of 84.4%, a sensitivity of 93.8%, and an AUC of 88.3%.

These results indicate that the AUC, accuracy, sensitivity, and specificity are high for the urinary creatinine-corrected urinary L-FABP concentrations on admission both for discrimination of patients who experienced aggravation and for discrimination of patients who did not experience aggravation (remained mild or so) among the SARS-CoV-2-positive patients, similar to the results of ROC analysis shown in FIGS. 1(c) and 1(d) of the urinary L-FABP concentrations on admission not corrected with urinary creatinine concentration. This suggests that without being corrected with urinary creatinine concentration (namely, with no elimination of the influence of urine concentration), the urinary L-FABP concentration will help to assess the aggravation risk of COVID-19 with high accuracy, high sensitivity, and high specificity.

FIG. 3(f) is a graph showing the urinary creatinine-corrected urinary L-FABP concentrations (μg/gCre) on admission of the 58 SARS-CoV-2-positive patients, in which two points at the same urinary L-FABP concentration plotted on the left (on admission) and the right (after one week) are of the same positive patient. In FIG. 3(f), the points of the same positive patient plotted on the left (on admission) and the right (after one week) are both shaped (◯: severe, □: moderate, ∇: mild) to indicate the degree of severity at one week after admission. In FIG. 3(f), different types of hatching (patterns) are used to distinguish the severity on admission (left) and the degree of severity at one week after admission (right). The change between the degree of severity on admission and that after one week can be understood by comparing the hatching types of the points of the same positive patient plotted on the left (on admission) and the right (after one week). In FIG. 3(f), the cutoff point value for severe case is 22 (μg/gCre) from FIG. 3(c) and that for mild case is 9 (μg/gCre) from FIG. 3(d). The results plotted in FIG. 3(f) indicate that patients with a urinary creatinine-corrected urinary L-FABP concentration on admission higher than or equal to a cutoff point value of 22 μg/gCre have a significantly strong tendency to experience aggravation from mild to moderate or severe or from moderate to severe in one week. On the other hand, the results plotted in FIG. 3(f) suggest that patients with a urinary creatinine-corrected urinary L-FABP concentration on admission lower than or equal to a cutoff point value of 9 μg/gCre are mild on admission and still mild after one week and have no aggravation risk or a low aggravation risk except for few cases. These results are similar to those shown in FIG. 1(f) with respect to the urinary L-FABP concentration not corrected with urinary creatinine concentration. Thus, with or without being corrected with urinary creatinine concentration, the urinary L-FABP concentration will help to assess the aggravation risk of COVID-19.

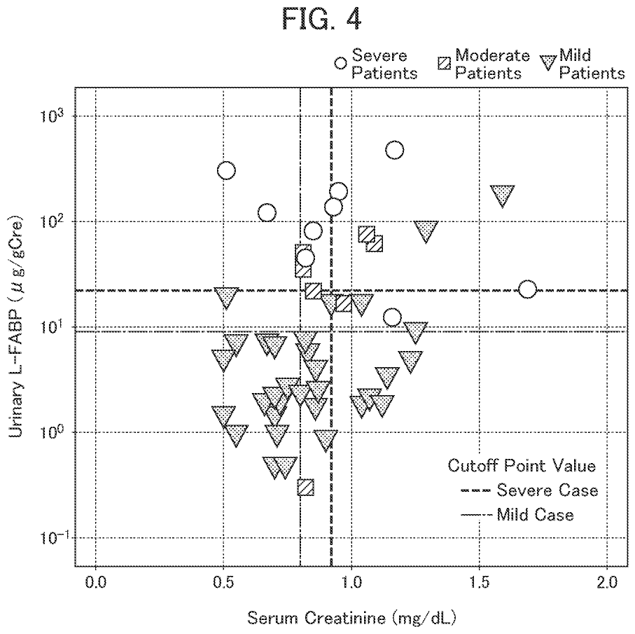

Next, a two-dimensional plot was made in which the urinary creatinine-corrected urinary L-FABP concentrations on admission of the 58 SARS-CoV-2-positive patients were on the vertical axis with the point shape and the hatching type indicating the degree of severity at one week after admission (corresponding to the right plot in FIG. 3(f)), and the serum creatinine concentrations on admission of the 58 SARS-CoV-2-positive patients were on the horizontal axis with the point shape and the hatching type indicating the degree of severity at one week after admission (corresponding to the right plot in FIG. 1(e) or 3(e)). This further clarified whether the aggravation risk of SARS-CoV-2 correlated with the urinary L-FABP concentration (corrected with urinary creatinine concentration) or the serum creatinine concentration. The results are shown in FIG. 4.

The results plotted in FIG. 4 indicate that patients with a urinary creatinine-corrected urinary L-FABP concentration on admission higher than or equal to a cutoff point value of 22 μg/gCre have a significantly strong tendency to experience aggravation in one week. On the other hand, the results plotted in FIG. 4 indicate no correlation between the serum creatinine concentration on admission and aggravation whether or not it exceeds the cutoff point value 0.92 mg/dL.

### Example 3

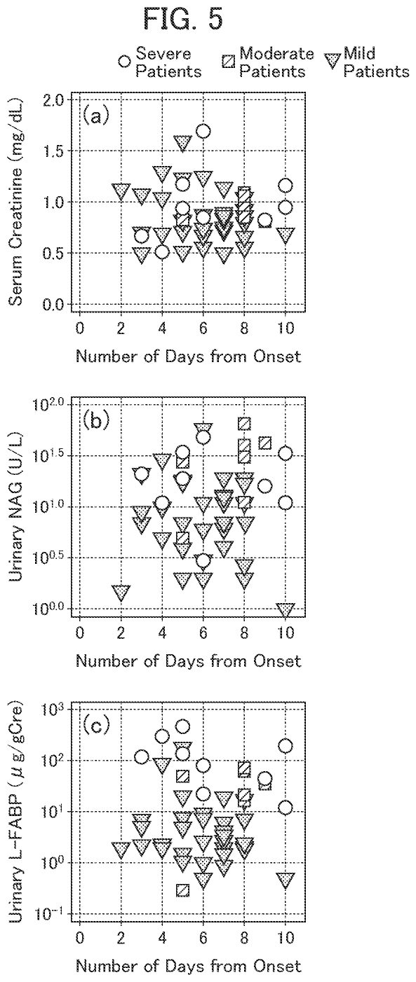

With respect to the 58 SARS-CoV-2-positive patients, the serum creatinine concentration (mg/dL) on admission, the urinary N-acetyl-Q-D-glucosaminidase (NAG) concentration (U/L) on admission, and the urinary creatinine-corrected urinary L-FABP concentration (μg/gCre) on admission were each plotted against the number of days from onset to admission (to the measurement of the concentration). NAG is a marker enzyme present in renal tissue cells. In general, urinary NAG concentration (mg/dL) is considered an indicator of renal tissue injury. The urinary NAG concentration was measured according to the method described in the literature: Japanese Journal of Clinical Medicine, Vol. 43, Fall Extra Edition, pp. 234-236, 1985. The results are shown in FIGS. 5(a) to 5(c). In the FIGS. 5(a) to 5(c), each point plotted for each patient is shaped (◯: severe, □: moderate, ∇: mild) and hatched to indicate the degree of severity at one week after admission.

The results shown in FIG. 5(a) indicate no correlation between the serum creatinine concentration on admission and aggravation. Similarly, the results shown in FIG. 5(b) indicate no correlation between the urinary NAG concentration on admission and aggravation. On the other hand, the results shown in FIG. 5(c) indicate that the urinary L-FABP concentration will help to assess the aggravation risk one week ahead of the measurement independently of the time point at which the urinary L-FABP concentration is measured, as long as it is within 2 to 10 days from onset.

### Example 4

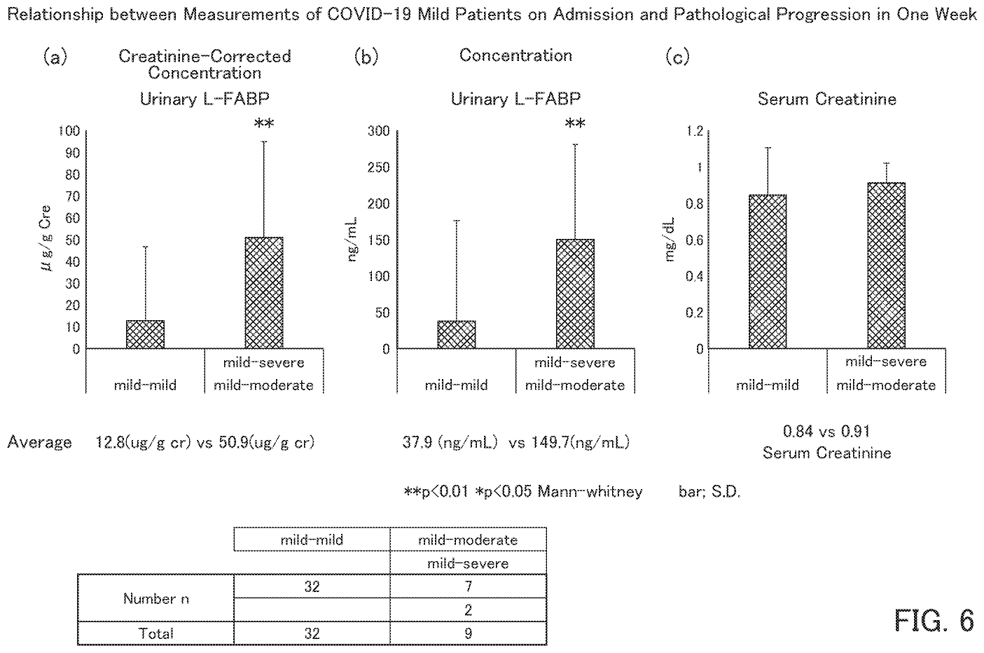

Forty-one SARS-CoV-2-positive mild patients were subjected on admission to measurement of creatinine-corrected urinary L-FABP concentration (μg/gCre), urinary L-FABP concentration (ng/mL), and serum creatinine concentration (mg/dL) as shown above. After one week after admission, the 41 patients, who were mild on admission, were evaluated for the degree of severity (mild, moderate, or severe). The results are shown in FIGS. 6(a) to 6(c).

FIGS. 6(a) to 6(c) are graphs showing correlation between the marker levels measured on admission of the 41 mild patients and the pathological progression in one week. In FIGS. 6(a) to 6(c), among the 41 patients who were mild on admission, those who were still mild after one week are indicated by “mild-mild”. Among the 41 patients who were mild on admission, those who became moderate in one week are indicated by “mild-moderate”. Among the 41 patients who were mild on admission, those who became severe in one week are indicated by “mild-severe”.

The results in FIGS. 6(a) to 6(c) show that, of the 41 patients who were mild on admission, 32 were still mild after one week (mild-mild). Of the 41 patients who were mild on admission, 7 became moderate in one week (mild-moderate). Of the 41 patients who were mild on admission, 2 became severe in one week (mild-severe).

FIG. 6(a) is a graph showing the average of the creatinine-corrected urinary L-FABP concentrations on admission of the 32 “mild-mild” patients and showing the average of the creatinine-corrected urinary L-FABP concentrations on admission of the “mild-moderate” and “mild-severe” patients (9 in total). The results in FIG. 6(a) show that the average of the creatinine-corrected urinary L-FABP concentrations of the “mild-mild” patients is 12.8 μg/gCre while the average of the creatinine-corrected urinary L-FABP concentrations of the “mild-moderate” and “mild-severe” patients is 50.9 μg/gCre. The results indicate that the creatinine-corrected urinary L-FABP concentrations on admission of the mild patients who became moderate or severe in one week are significantly higher, with p-value<0.01, than those on admission of the patients who were still mild after one week.

FIG. 6(b) is a graph showing the average of the urinary L-FABP concentrations on admission of the 32 “mild-mild” patients and showing the average of the urinary L-FABP concentrations on admission of the “mild-moderate” and “mild-severe” patients (9 in total). The results in FIG. 6(b) show that the average of the urinary L-FABP concentrations of the “mild-mild” patients is 37.9 ng/mL while the average of the urinary L-FABP concentrations of the “mild-moderate” and “mild-severe” patients is 149.7 ng/mL. The results indicate that the urinary L-FABP concentrations on admission of the mild patients who became moderate or severe in one week are significantly higher, with p-value<0.01, than those on admission of the patients who were still mild after one week.

FIG. 6(c) is a graph showing the average of the serum creatinine concentrations (mg/dL) on admission of the 32 “mild-mild” patients and showing the average of the serum creatinine concentrations (mg/dL) on admission of the “mild-moderate” and “mild-severe” patients (9 in total). The results in FIG. 6(c) show that the average of the serum creatinine concentrations of the “mild-mild” patients is 0.84 mg/dL while the average of the serum creatinine concentrations of the “mild-moderate” and “mild-severe” patients is 0.91 mg/dL. The results indicate that there is no significant difference between the serum creatinine concentrations on admission of the mild patients who became moderate or severe in one week and the serum creatinine concentrations on admission of the mild patients who were still mild after one week.

### Example 5

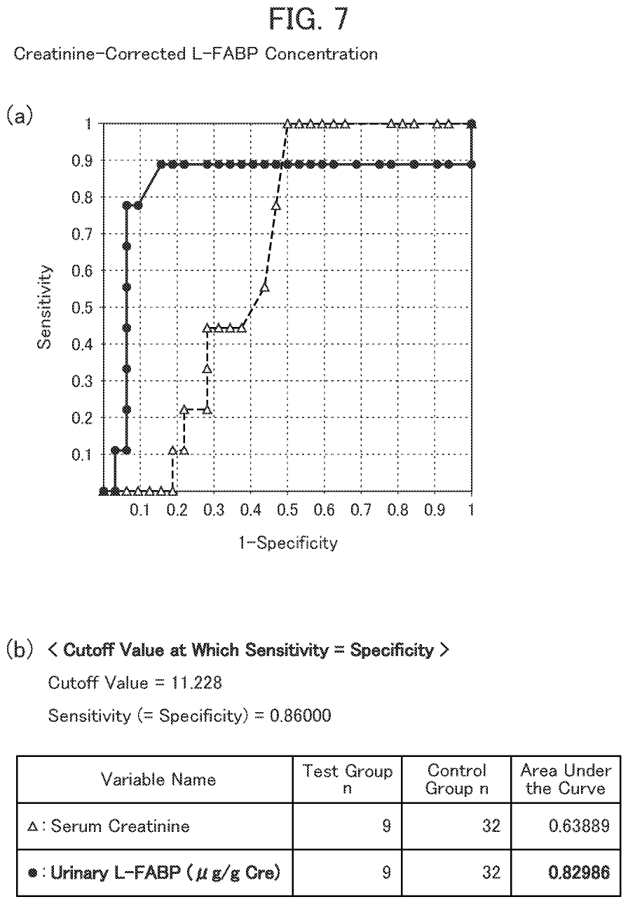

For discrimination between the “mild-mild” patients (32 patients of a control group) and the “mild-moderate” and “mild-severe” patients (9 patients of a test group), ROC analysis was performed on the creatinine-corrected urinary L-FABP concentrations (and the serum creatinine concentrations for comparison) on admission. The results are shown in FIGS. 7(a) and 7(b).

FIGS. 7(a) and 7(b) are a graph and a table showing the results of ROC analysis of the creatinine-corrected urinary L-FABP concentrations on admission (and the serum creatinine concentrations for comparison) of the “mild-moderate” and “mild-severe” patients versus the “mild-mild” patients. The results of ROC analysis shown in FIGS. 7(a) and 7(b) indicate that a cutoff value (the point at which sensitivity equals specificity) of 11.228 (μg/gCre) was obtained for the creatinine-corrected urinary L-FABP concentrations on admission of the “mild-moderate” and “mild-severe” patients, with an AUC as high as 0.82986 (82.986%). For the serum creatinine concentrations, AUC was as low as 0.63889 (63.889%). The results suggest that the measurement of the creatinine-corrected urinary L-FABP concentration on admission will help to discriminate, with high accuracy, patients who are mild on admission but have an aggravation risk from mild to moderate or severe in one week.

### Example 6

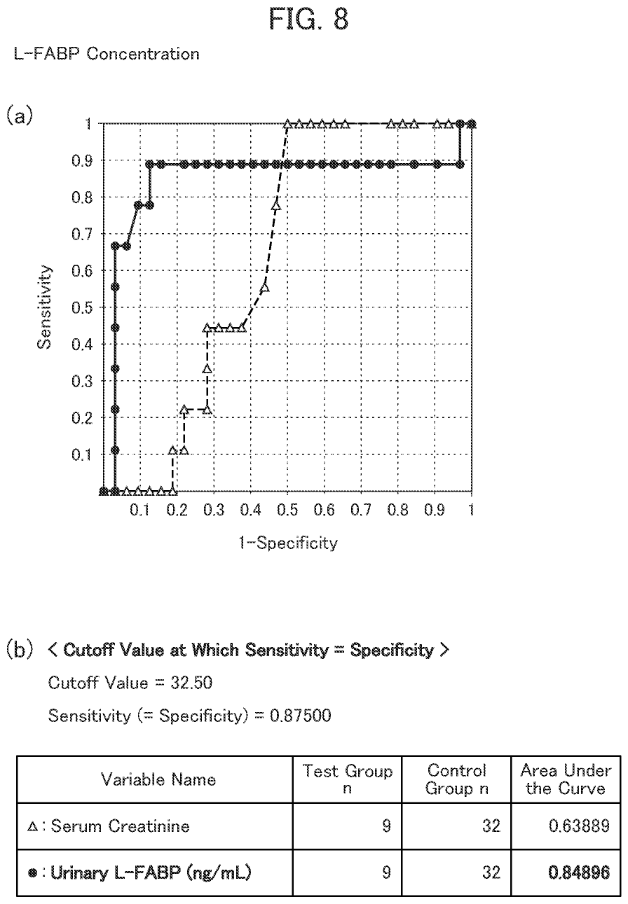

For discrimination between the “mild-mild” patients (32 patients of a control group) and the “mild-moderate” and “mild-severe” patients (9 patients of a test group), ROC analysis was performed on the urinary L-FABP concentrations (and the serum creatinine concentrations for comparison) on admission. The results are shown in FIGS. 8(a) and 8(b).

FIGS. 8(a) and 8(b) are a graph and a table showing the results of ROC analysis of the urinary L-FABP concentrations on admission (and the serum creatinine concentrations for comparison) of the “mild-moderate” and “mild-severe” patients versus the “mild-mild” patients. The results of ROC analysis shown in FIGS. 8(a) and 8(b) indicate that a cutoff value (the point at which sensitivity equals specificity) of 32.50 ng/mL was obtained for the urinary L-FABP concentrations on admission of the “mild-moderate” and “mild-severe” patients, with an AUC as high as 0.84896 (84.896%). For the serum creatinine concentrations, AUC was as low as 0.63889 (63.889%). The results suggest that the measurement of the urinary L-FABP concentration on admission will help to discriminate, with high accuracy, patients who are mild on admission but have an aggravation risk of from mild to moderate or severe in one week.

### SEQUENCE LISTING

ATF-488PCT 4T25.tzt:

1. A method of assessing an aggravation risk of SARS-CoV-2 infectious disease (COVID-19), the method comprising:
quantifying a liver-type fatty acid binding protein in urine collected from a subject; and
assessing an aggravation risk of SARS-CoV-2 infectious disease (COVID-19) based on a result of the quantification.

2. The method according to claim 1, wherein the quantification is performed at least 2 times at specific intervals of days.

3. A COVID-19 aggravation risk assessment kit for use in the method according to claim 1, the kit comprising a material for quantifying a liver-type fatty acid binding protein.

4. The COVID-19 aggravation risk assessment kit according to claim 3, wherein the material for quantifying a liver-type fatty acid binding protein is an anti-L-FABP antibody.

5. A companion diagnostic agent comprising a material for quantifying a liver-type fatty acid binding protein, the companion diagnostic agent being for use in selecting a therapeutic or prophylactic drug for COVID-19 using the method according to claim 1.

6. A COVID-19 aggravation risk marker comprising a liver-type fatty acid binding protein and being for use as a target to be quantified in the method according to claim 1.

7. A companion diagnostic method for COVID-19, comprising: performing the COVID-19 aggravation risk assessment method according to claim 1; and performing at least one selection selected from the group consisting of: selecting a treatment for the subject depending on the aggravation risk determined by the assessment method; and selecting a therapeutic or prophylactic drug for COVID-19 depending on the aggravation risk determined by the assessment method.

8. The companion diagnostic method according to claim 7, wherein, when the subject has a mild condition and is determined to still have a mild condition specific days or more ahead by the assessment method, the treatment comprises at least one treatment selected from the group consisting of: watch-and-wait, isolation, administration of commercially available cold medicines and hydration.

9. The companion diagnostic method according to claim 7, wherein, when determined to have an aggravation risk from mild to moderate or severe or from moderate to severe within specific days by the assessment method, the treatment comprises at least one treatment selected from the group consisting of: hospitalization, ICU transfer, antibiotic administration against intercurrent microbial infection, oxygen supplementation, high-flow nasal cannula oxygen therapy, noninvasive or invasive mechanical ventilation, extracorporeal membrane oxygenation (ECMO), acute blood purification, and hemoadsorption therapy.

10. The companion diagnostic method according to claim 7, wherein the therapeutic or prophylactic drug for COVID-19 comprises at least one drug selected from the group consisting of antiviral drugs, cytokine storm- or acute respiratory distress syndrome (ARDS)-ameliorating drugs, and vaccines and antibodies.

11. A therapeutic or prophylactic method for COVID-19, comprising: performing the COVID-19 aggravation risk assessment method according to claim 1; and performing at least one selected from the group consisting of: performing a treatment for the subject depending on the aggravation risk determined by the assessment method; and administering, to the subject, a therapeutic or prophylactic drug for COVID-19 depending on the aggravation risk determined by the assessment method.

12. The therapeutic or prophylactic method according to claim 11, wherein, when the subject has a mild condition and is determined to still have a mild condition specific days or more ahead by the assessment method, the treatment comprises at least one treatment selected from the group consisting of: watch-and-wait, isolation, administration of commercially available cold medicines and hydration.

13. The therapeutic or prophylactic method according to claim 11, wherein, when determined to have an aggravation risk from mild to moderate or severe or from moderate to severe within specific days by the assessment method, the treatment comprises at least one treatment selected from the group consisting of: hospitalization, ICU transfer, antibiotic administration against intercurrent microbial infection, oxygen supplementation, high-flow nasal cannula oxygen therapy, noninvasive or invasive mechanical ventilation, extracorporeal membrane oxygenation (ECMO), acute blood purification, and hemoadsorption therapy.

14. The therapeutic or prophylactic method according to claim 11, wherein the therapeutic or prophylactic drug for COVID-19 comprises at least one drug selected from the group consisting of antiviral drugs, cytokine storm- or acute respiratory distress syndrome (ARDS)-ameliorating drugs, and vaccines and antibodies.
